(19) United States
(12) Patent Application Publication
(10) Pub. No.: US 2009/0062520 A1
(43) Pub. Date: Mar. 5, 2009

(54) Method of producing DNA structure and DNA structure

(76) Inventors: Naoki Sugimoto, Kobe-shi (JP); Daisuke Miyoshi, Kobe-shi (JP); Mamiko Inoue, Machida-shi (JP)

Correspondence Address: MCDERMOTT WILL & EMERY LLP, 600 13TH STREET, NW, WASHINGTON, DC, 20005-3096, US

(21) Appl. No.: 11/896,612

(22) Filed: Sep. 4, 2007

(51) Int. Cl. C07H 21/04

(52) U.S. Cl. 536/23.1

(57) ABSTRACT

The present invention provides a method of producing a DNA structure in which multiple quadruplex DNAs are linked, which includes (a) a step of mixing multiple DNA molecules having an antiparallel quadruplex structural part, and at least two single stranded sticky ends extended from the end of the quadruplex structural part, wherein the single stranded sticky end of the each DNA molecule has a base sequence that can form a duplex through interaction with the single stranded sticky end of other DNA molecule.

### BACKGROUND OF THE INVENTION

1. Field of the Invention

The present invention relates to a method of producing a DNA structure by linking DNA molecules, and a DNA structure.

2. Description of Related Art

Techniques for densification and integration of semiconductor elements mainly including silicon have advanced in accordance with Moore's Law proposed by Gordon Moore (that the number of chips that can be integrated in transistors increases through doubling approximately every two years), and have greatly contributed to speeding up and sophistication of data processing, and to increase in communication capacity, globalization of network area, and the like in recent years. Whereas, however, the accuracy of their microprocessing has been almost reaching to the limit. The size of the transistor attainable at a current mass production level has reached to approximately 50 nm, and further accuracy in the processing to attain the size of 20 nm will have come to be demanded in the next ten years. However, achievement of such a goal is considered to be difficult in both technical and economical aspects.

Under such circumstances, a technique referred to as bottom-up nano-technology have been investigated recently. This technique is based on an approach completely different from that of top-down type technique “to fabricate fine pattern through processing of the material” typified by photolithography which had been in progress in the field of semiconductors, and is expected to enable microprocessing that cannot be achieved by the top-down type technique. Fundamental feature of the bottom-up nano-technology is that molecules or atoms having a size in the order of nm or lower are built up into an intended structure in a self-organizing manner utilizing the chemical bond or the intermolecular force originally carried by them, and studies have been carried out predominantly on biomolecules or molecules that mimic such biomolecules. Among them, DNAs are extremely interesting in characteristics capable of forming a variety of desired higher order structures in a self-organizing manner by the design of the sequence based on the formation of base pairs involving A (adenine)/T (thymine), G (guanine)/C (cytosine). For example, Winfree, E., et al., Nature 394 (1998) 539-544 describes a method of forming a two-dimensional DNA structure having a stripe pattern through utilizing duplex formation among multiple single stranded DNAs. Furthermore, Rothemund, P. W. K., Nature 440 (2006) 297-302 describes that complicated patterns such as shapes of snow crystals and American Continent could be formed with DNAs. Since such techniques for controlling the complicated higher order structures are believed to be unavailable with any molecules other than DNAs, DNAs have been greatly expected for the bottom-up nano-technologies.

Many of higher order structure of DNAs which have been developed hitherto have a duplex structure mainly as the skeleton. The duplex structure of DNAs is a most well known DNA structure, and the technique for anticipating the duplex structure has been established. Therefore, it is deemed that formation of a complicated structure using it as the skeleton would be comparatively easy. However, the duplex structure is very soft, and has no function per se. Accordingly, the higher order structures of DNAs formed based on the duplex structure serve as just merely unstable frameworks or foundations, and any laborious treatment such as introduction of a functionalized artificial and modified nucleic acid would be required for imparting a function to these structures.

On the other hand, there have existed quadruplex structures of DNAs as a functional DNA structure expected to be capable of excluding the need for such a treatment. FIG. 24 shows schematic drawings illustrating a quadruplex structure of a DNA. Conventionally known quadruplex structure of the DNA is formed by association of four DNA chains 1 rich in G bases 2 in a self-organizing manner, as shown in FIG. 24(a), to provide a quadruplex structure 3 as shown in FIG. 24(b). The quadruplex structure 3 is greatly different from the duplex DNA structure formed on the basis of Watson-Crick base pairs, i.e., A/T, G/C base pairs. More specifically, the quadruplex structure 3 is formed via hydrogen bondings of four G bases 2 as shown in FIG. 24(c) to give a structure referred to as a G-quartet, and the structure is retained by π-π stacking interaction among the G-quartet faces 6. In addition, although not shown in the figure, coordination of a metal ion between the G-quartet faces is required for formation of the quadruplex DNA structure, and the coordination of a K ion, a Na ion or the like has been known.

FIG. 25 shows drawings illustrating the conventionally known DNA quadruplex structures with categorization based on their patterns of formation. As shown in FIG. 25, several patterns of the quadruplex DNAs have been known heretofore. The quadruplex DNAs can be first categorized broadly into the following two types: DNA 4 in which 5′ to 3′ orientation of each four DNA chains 81 that form G-quartet faces 6 is identical (the 5′ to 3′ orientation being indicated by an arrow in the figure for facilitating the understanding) as shown in FIG. 25(a); and DNAs 7 and 8 in which two chains among the four chains are oriented oppositely to the remaining two chains as shown in FIGS. 25(b) and (c). Herein, the former is referred to as “parallel quadruplex DNA” or “parallel quadruplex structure”, and the latter is referred to as “antiparallel quadruplex DNA” or “antiparallel quadruplex structure”. These can be further categorized into subgroups. For example, the antiparallel quadruplex DNAs include DNA 7 formed by assembly of two molecules of single stranded DNA 9 of the same kind as shown in FIG. 25(b), and DNA 8 formed by intramolecular interaction caused in one single stranded DNA 82 as shown in FIG. 25(c). Herein, the former is referred to as “intermolecular antiparallel quadruplex DNA” or “intermolecular antiparallel quadruplex structure”, and the latter is referred to as “intramolecular antiparallel quadruplex DNA” or “intramolecular antiparallel quadruplex structure”.

FIG. 26 shows a drawing illustrating steps of formation of the intermolecular antiparallel quadruplex DNA. Two chains of the single stranded DNA 9 as shown in FIG. 26(a) are associated as shown in FIG. 26(b) to form the intermolecular antiparallel quadrupled DNA 7 as shown in FIG. 26(c). Each single stranded DNA 9 includes at least: two sequences 10 that participate in formation of the G-quartet face (hereinafter, the sequence thus participating in formation of the G-quartet face is referred to as “quadrupled structure forming sequence”.), and a sequence 11 that is positioned between the sequences 10 and forms a loop structure through folding of the single stranded DNA 9 in assembly of the intermolecular quadruplex DNA 7 (hereinafter, the sequence that forms a loop structure in assembly of the quadruplex DNA is referred to as “loop structure forming sequence”.) Therefore, two molecules of the single stranded DNA 9 satisfying these features are associated to result in folding at a part of the loop structure forming sequence 11, and thus the intermolecular quadruplex DNA 7 is stabilized via formation of the G-quartet faces 6 and π-π stacking interactions among these G-quartet faces 6 which are provided with four quadruplex structure forming sequences 10 in total that are present in each molecule. For retention of the structure of the quadruplex DNA 7, at least two or more G-quartet faces 6 are needed. Accordingly, at least two or more G bases must be included in the quadruplex structure forming sequence 10 as well. In addition, it is necessary that the loop structure forming sequence 11 has usually three or more bases so as not to inhibit formation of the G-quartet face 6.

Examples of the base sequence which have been demonstrated to form the intermolecular antiparallel quadruplex DNA so far include [d(G₃T₄G₃)] (SEQ ID NO: 13), [d(G₄T₄G₄)] (SEQ ID NO: 14), [d(G₃CT₄G₃C)] (SEQ ID NO: 15), [d(GCG₂T₃GCG₂)] (SEQ ID NO: 16), [d(A₂G₂T₄A₂G₂)] (SEQ ID NO: 17), [d(G₃T₂CAG₂)] (SEQ ID NO: 18), and the like.

FIG. 27 shows a drawing illustrating steps of formation of the intramolecular antiparallel quadruplex DNA. A single stranded DNA 82 as shown in FIG. 27(a) associates intramolecularly as shown in FIG. 27(b) to form the intramolecular antiparallel quadruplex DNA 8 as shown in FIG. 27(c). The single stranded DNA 82 has a structure in which four quadruplex structure forming sequences 10 and three loop structure forming sequences 11 are alternately repeated, and the G-quartet faces 6 are formed among the intramolecular quadruplex structure forming sequences 10. Also in the case of the intramolecular antiparallel quadruplex DNA 8, at least two or more G-quartet faces 6 are required for stabilizing the structure, similarly to the intermolecular antiparallel quadruplex DNA 7. Therefore, also the quadruplex structure forming sequence 10 here must include two or more guanine residues. In addition, also in connection with the loop structure forming sequence 11, it is necessary to have usually two or more bases so as not to inhibit formation of the G-quartet face 6. Examples of the base sequences which have been demonstrated to form the intramolecular antiparallel quadruplex DNA so far include [d(G₄T₄)₃G₄] (SEQ ID NO: 19), [d(T₂G₄)₄] (SEQ ID NO: 20), [d(G₂T₂G₂TGTG₂T₂G₂)] (SEQ ID NO: 21), [dAG₃(T₂AG₃)₃] (SEQ ID NO: 22), and the like.

The quadruplex DNA was proven to have various functions due to its specific structure which are not found in the case of the duplex structure. For example, as described above, metal ions such as K ion and Na ion can be arranged in the structure of the quadruplex DNA. Furthermore, it is known that some molecules such as anthraquinone, acridine, porphyrin derivatives may be intercalated in the quadruplex DNA (see, Sun, D. et al., J. Med. Chem 40 (1997) 2113-2118; and Wheelhouse, R. T. et al., J. Am. Chem. Soc. 120 (1998) 3261-3262). Particularly, in the case of porphyrin, the quadruplex DNA serves as a reaction field for allowing Zn²⁺ to form a coordinate bond in porphyrin.

Furthermore, although DNAs have ascertained to have hole transport ability, the base playing its central role has been considered to be the G base having the lowest oxidation potential. Therefore, the quadruplex DNA having a structure with the G bases at a high density has been expected to yield a nanowire having a high hole transport property. Moreover, there are two types of the quadruplex DNAs, i.e., parallel type and antiparallel type, as described above. Because these two different structures are switched depending on changes in the eternal environment (type of metal ion, addition of PEG, change in pH, and the like), their applications to nano switching devices have been also suggested. For example, Japanese Unexamined Patent Application Publication No. 2006-316004 discloses a novel bipyridine-modified quadruplex DNA switch which can control the antiparallel and parallel structure transition depending on the presence of a metal having valency of two or more. In addition, the quadruplex DNAs are characterized by higher rigidity and stability to heat as compared with duplex DNAs.

### SUMMARY OF THE INVENTION

As in the foregoing, the quadruplex DNAs are very hopeful as a functional nano material, and attractive in terms of their wide variety of functions, in particular. Thus, it is expected that attainment of more complicated nanostructures including the quadruplex DNA, for example, multifunctional nanomaterials in which quadruplex DNAs having different functions are linked (for example, any combination of a quadruplex DNA having a coordinated K ion, and a quadruplex DNA having a coordinated Na ion, and the like) will provide important techniques in future bottom-up nano-technologies. To this end, a technique for linking two kinds of quadruplex DNAs which were independently formed is essential.

The present invention was made in view of such circumstances, and an object of the invention is to provide a method of producing a DNA structure in which independently formed multiple quadruplex DNAs are linked.

The present inventors elaborately investigated to solve the aforementioned problems, and consequently succeeded in production of a DNA structure in which independently formed multiple quadruplex DNAs are linked. More specifically, the method of producing a DNA structure of the present invention is a method of producing a DNA structure in which multiple quadruplex DNAs are linked, which includes (a) a step of mixing multiple DNA molecules having an antiparallel quadruplex structural part, and at least two single stranded sticky ends extended from the end of the quadruplex structural part, wherein the single stranded sticky end of the each DNA molecule has a base sequence that can form a duplex through interaction with the single stranded sticky end of other DNA molecule.

In one embodiment of the aforementioned method of the production, the DNA molecules to be mixed in the step (a) include at least two kinds of molecules, i.e., a first DNA molecule and a second DNA molecule, wherein one single stranded sticky end of the first DNA molecule, and one single stranded sticky end of the second DNA molecule have a base sequence that can form a duplex through interaction, and wherein other single stranded sticky end of the first DNA molecule, and other single stranded sticky end of the second DNA molecule have a base sequence that can form a duplex through interaction.

In the aforementioned method, the first DNA molecule and the second DNA molecule may be a monomer DNA molecule having the quadruplex structural part each formed intramolecularly, or may be a dimer DNA molecule having the quadruplex structural part formed intermolecularly.

The single stranded sticky end is constructed so as to have a base sequence including preferably 10 or more bases. Moreover, the quadruplex structural part is preferably constructed so as to include two or more and five or less G-quartet faces formed by a hydrogen bonding among four guanine bases.

Furthermore, the DNA structure of the present invention is that multiple DNA molecules having an antiparallel quadruplex structural part are linked via a junction part, wherein: the DNA molecule has at least two single stranded sticky ends extended from the end of the quadruplex structural part; the single stranded sticky end of the each DNA molecule has a base sequence that can form a duplex through interaction with the single stranded sticky end of other DNA molecule; and the junction part is a duplex formed through the interaction of the single stranded sticky ends of adjacent two DNA molecules.

The foregoing object, other object, features and advantages of the present invention will be apparent from the detailed description of preferred embodiments below, with reference to accompanying drawings.

### BRIEF DESCRIPTION OF THE DRAWINGS

FIG. 1 shows a drawing illustrating steps of forming a first DNA molecule having an intermolecular antiparallel quadruplex structural part in Embodiment 1.

FIG. 2 shows a drawing illustrating steps of forming a second DNA molecule having an intermolecular antiparallel quadruplex structural part in Embodiment 1.

FIG. 3 shows a drawing illustrating steps of forming a DNA structure through alternately linking two kinds of the intermolecular antiparallel quadrupled DNAs in Embodiment 1.

FIG. 4 shows a schematic drawing illustrating a method of obtaining a DNA structure having a desired length by alternately linking two kinds of the intermolecular antiparallel quadruplex DNAs in Embodiment 1.

FIG. 5 shows a schematic drawing illustrating a method of obtaining a DNA structure by linking three kinds of the intermolecular antiparallel quadruplex DNAs in Embodiment 1.

FIG. 6 shows a drawing illustrating steps of forming a first DNA molecule having an intramolecular antiparallel quadrupled structural part in Embodiment 2.

FIG. 7 shows a drawing illustrating steps of forming a second DNA molecule having an intramolecular antiparallel quadruplex structural part in Embodiment 2.

FIG. 8 shows a drawing illustrating steps of forming a DNA structure through alternately linking two kinds of the intramolecular antiparallel quadrupled DNAs in Embodiment 2.

FIG. 9 shows a drawing illustrating steps of forming a DNA structure through linking each one molecule of two kinds of the intramolecular antiparallel quadruplex DNAs in Embodiment 2.

FIG. 10 shows a schematic drawing illustrating a method of obtaining a DNA structure having a desired length by alternately linking two kinds of the intramolecular antiparallel quadruplex DNAs in Embodiment 2.

FIG. 11 shows a schematic drawing illustrating an experimental sample in Comparative Example 1.

FIG. 12 shows a drawing illustrating results of native gel electrophoresis in Comparative Example 1.

FIG. 13 shows a schematic drawing illustrating an experimental sample in Comparative Example 2.

FIG. 14 shows a drawing illustrating results of native gel electrophoresis in Comparative Example 2.

FIG. 15 shows a drawing illustrating results of CD spectra in Example 1.

FIG. 16 shows a drawing illustrating results of a CD difference spectrum in Example 1.

FIG. 17 shows a drawing illustrating results of native gel electrophoresis in Example 1.

FIG. 18 shows a drawing illustrating results of CD spectra in Example 2.

FIG. 19 shows a drawing illustrating results of a CD difference spectrum in Example 2.

FIG. 20 shows a drawing illustrating results of native gel electrophoresis in Example 2.

FIG. 21 shows a schematic drawing illustrating an experimental sample in Example 3.

FIG. 22 shows a drawing illustrating results of native gel electrophoresis in Example 3.

FIG. 23 shows a drawing illustrating results of native gel electrophoresis in Example 4.

FIG. 24 shows a schematic drawing illustrating a structure of a conventional quadruplex DNA.

FIG. 25 shows a drawing for explaining categorization of conventional quadruplex DNAs.

FIG. 26 shows a drawing illustrating steps of forming a conventional intermolecular antiparallel quadruplex DNA.

FIG. 27 shows a drawing illustrating steps of forming a conventional intramolecular antiparallel quadruplex DNA.

### DETAILED DESCRIPTION OF PREFERRED EMBODIMENTS

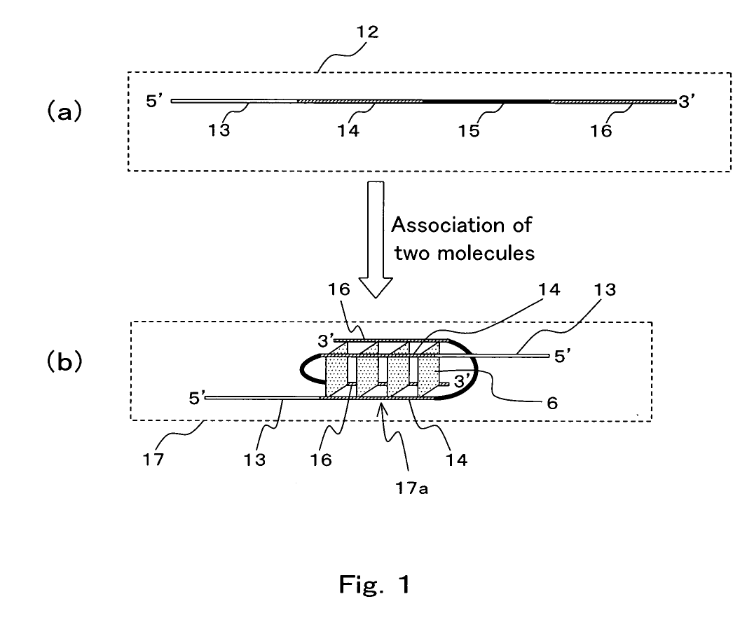

Hereinafter, modes for carrying out the present invention will be explained with reference to FIG. 1 to FIG. 10.

### Embodiment 1

In Embodiment 1, a DNA structure in which two kinds of DNA molecules (a first DNA molecule and a second DNA molecule) having an intermolecular antiparallel DNA quadruplex structure are linked, and a method of the production thereof are explained.

FIG. 1 shows a drawing illustrating steps of forming a first DNA molecule having an intermolecular antiparallel quadruplex structural part. Two molecules of DNA 12 as shown in FIG. 1(a) associate to form a first DNA molecule 17 having an intermolecular antiparallel quadruplex structural part 17a as shown in FIG. 1(b). In the first DNA molecule 17, duplex structure forming sequences 13 in an unstructured state are extended from two ends of the quadruplex structural part 17a. The first DNA molecule 17 is a dimer DNA molecule. As shown in FIG. 1(a), each DNA 12 is constructed to include, from 5′ end, a duplex structure forming sequence 13, a quadruplex structure forming sequence 14, a loop structure forming sequence 15 and a quadruplex structure forming sequence 16, which are arranged in this order. Two molecules of the DNA 12 having such a construction are assembled to allow the quadruplex structure forming sequences 14 and 16 to interact, whereby the antiparallel quadruplex structural part 17a is formed to result in formation of the first DNA molecule 17 including the same. In the antiparallel quadruplex structural part 17a, four G bases form a G-quartet face 6 via hydrogen bondings, and π-π stacking interaction of the G-quartet faces 6 are caused to retain the structure.

In the DNA 12, the base sequence of the quadruplex structure forming sequences 14 and 16 may be designed such that the antiparallel quadruplex structural part 17a is formed. To this end, it is necessary to design so as to form at least two G-quartet faces 6. However, it is more preferred to design so as to form two or more and five or less G-quartet faces 6, because the quadruplex structure forming sequences 14 and 16 are likely to form a parallel quadruplex structure when there are six or more G-quartet faces 6. Moreover, in the DNA 12, the loop structure forming sequence 15 is not particularly limited with respect to its base sequence and length, and may be designed so as not to inhibit formation of the quadruplex structural part 17a. Details of the duplex structure forming sequence 13 will be described herein later.

The base sequence of the DNA 12 may be constructed to include, from 5′ end, the quadruplex structure forming sequence 16, the loop structure forming sequence 15, the quadruplex structure forming sequence 14 and the duplex structure forming sequence 13 in this order, unlike the base sequence shown in FIG. 1(a). Additionally, the DNA 12 may also include a base sequence other than those described above.

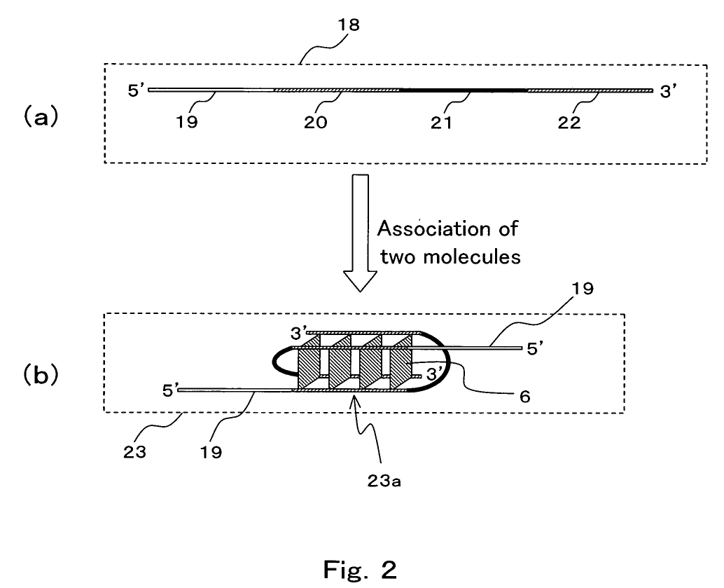

On the other hand, the second DNA molecule having the intermolecular antiparallel quadruplex structural part also has a construction similar to that of the aforementioned first DNA molecule 17. FIG. 2 shows a drawing illustrating steps of forming a second DNA molecule having an intermolecular antiparallel quadruplex structural part. Two molecules of DNA 18 as shown in FIG. 2(a) associate to form a second DNA molecule 23 having an intermolecular antiparallel quadruplex structural part 23a as shown in FIG. 2(b). In the second DNA molecule 23, duplex structure forming sequences 19 in an unstructured state are extended from two ends of the quadruplex structural part 23a. The second DNA molecule is a dimer DNA molecule. As shown in FIG. 2(a), each DNA 18 is constructed to include, from 5′ end, a duplex structure forming sequence 19, a quadruplex structure forming sequence 20, a loop structure forming sequence 21 and, a quadruplex structure forming sequence 22 which are arranged in this order. Two molecules of the DNA 18 having such a construction are assembled to allow the quadruplex structure forming sequences 20 and 22 to interact, whereby the antiparallel quadruplex structural part 23a is formed to result in formation of the second DNA molecule 23 including the same. In the antiparallel quadruplex structural part 23a, four G bases form a G-quartet face 6 via Hoogsteen hydrogen bondings, and π-π stacking interaction of the G-quartet faces 6 are caused to retain the structure.

In the DNA 18, the base sequence of the quadruplex structure forming sequences 20 and 22 may be designed such that the antiparallel quadruplex structural part 23a is formed. To this end, it is necessary to design so as to form at least two G-quartet faces 6. However, it is more preferred to design so as to form two or more and five or less G-quartet faces 6, because the quadruplex structure forming sequences 20 and 22 are likely to form a parallel quadruplex structure when there are six or more G-quartet faces 6. Moreover, in DNA 18, the loop structure forming sequence 21 is not particularly limited with respect to its base sequence and length, and may be designed so as not to inhibit formation of the quadruplex structural part 23a. Details of the duplex structure forming sequence 19 will be described herein later.

The base sequence of the DNA 18 may be constructed to include, from 5′ end, the quadruplex structure forming sequence 22, the loop structure forming sequence 21, the quadruplex structure forming sequence 20 and the duplex structure forming sequence 19 in this order, unlike the base sequence shown in FIG. 2(a). Additionally, the DNA 18 may also include a base sequence other than those described above.

The duplex structure forming sequence 13 of the DNA 12, and the duplex structure forming sequence 19 of the DNA 18 are designed complementary to each other. The duplex structure forming sequence 13 and the duplex structure forming sequence 19 are acceptable as long as they are designed such that they can form a duplex through their interaction, and thus they may not be necessarily complementary in the entire of these base sequences. The duplex structure forming sequence 13 and the duplex structure forming sequence 19 preferably has a base sequence including 10 or more bases in light of structure stability of the DNA structure constructed through linking the first DNA molecule 17 and the second DNA molecule 23.

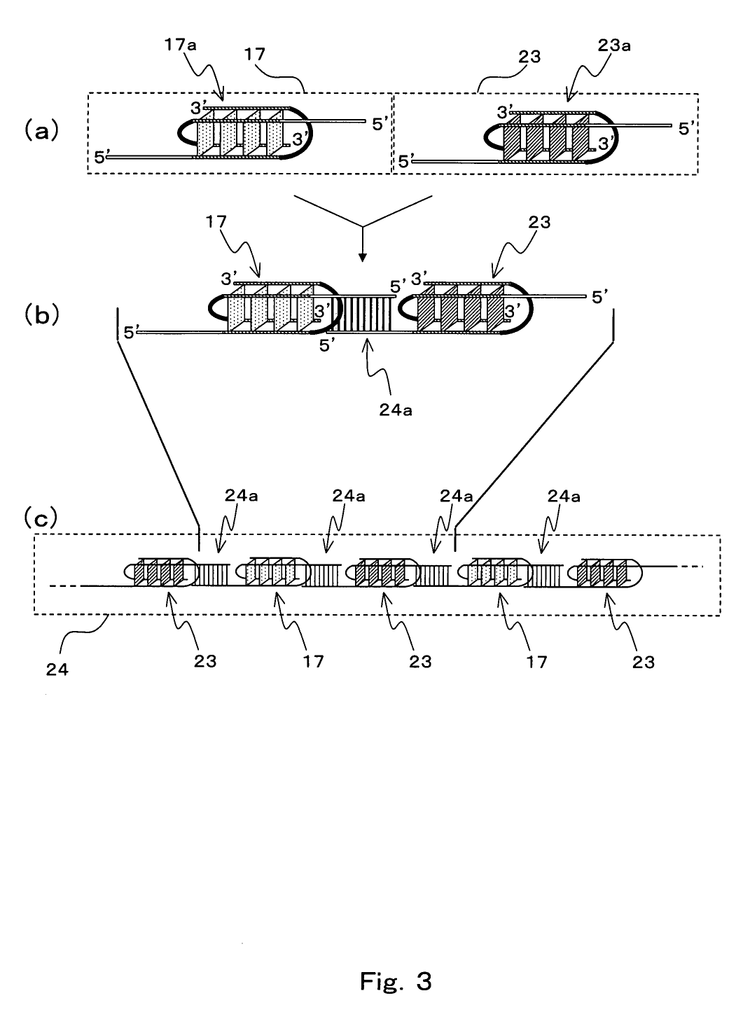

FIG. 3 shows a drawing illustrating steps of forming a DNA structure including a first DNA molecule and a second DNA molecule. When each independently formed first DNA molecule 17 and second DNA molecule 23 each having intermolecular antiparallel quadruplex structural part 17a or 23a as shown in FIG. 3(a) are mixed in an adequate solution, the first DNA molecule 17 and the second DNA molecule 23 are linked in a self-organizing manner via the duplex 24a formed by the interaction of the duplex structure forming sequences 13 and 19 to form a DNA structure 24, as shown in FIG. 3(c). The DNA structure 24 has a construction in which a plurality of the first DNA molecules 17 and the second DNA molecules 23 are linked in one orientation, thereby having a construction as a nanowire. FIG. 3(b) shows a partially enlarged view of the DNA structure 24.

As described above, when the independently formed first DNA molecules 17 and second DNA molecules 23 are merely mixed in the same solution, the DNA structure 24 having a variety of length with both molecules alternately linked would be obtained.

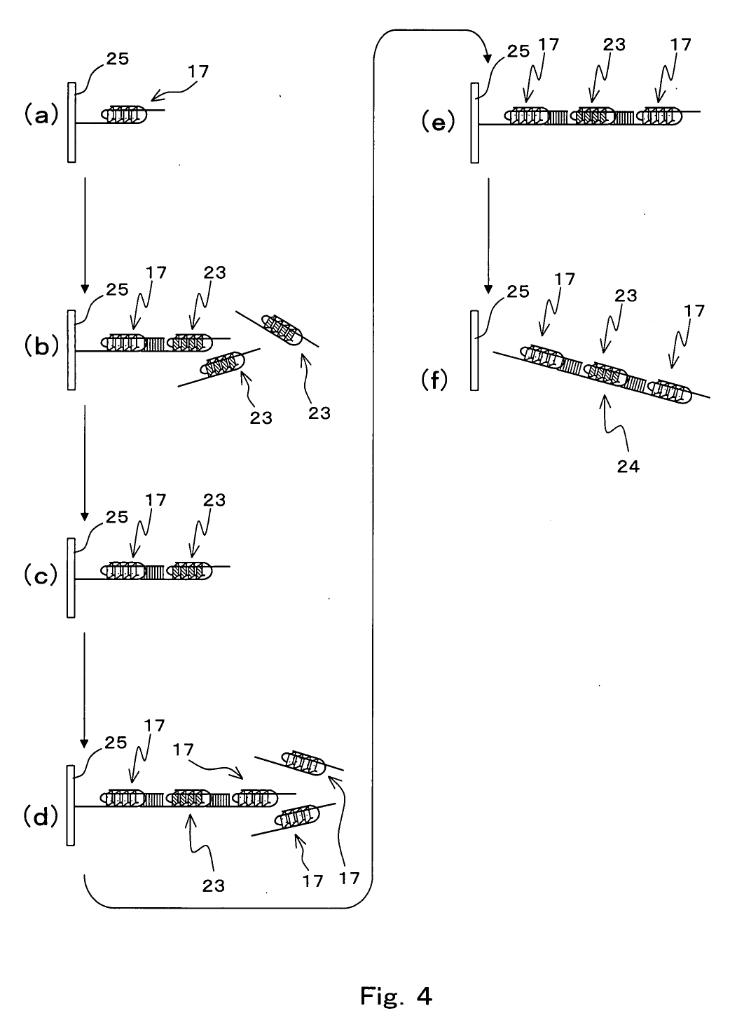

FIG. 4 shows a schematic drawing illustrating a method of obtaining a DNA structure (nanowire) having a desired length by alternately linking a first DNA molecule and a second DNA molecule each having the intermolecular antiparallel quadruplex structural part. As shown in FIG. 4(a), the first DNA molecule 17 is first adsorbed beforehand on a substrate 25 such as gold by a conventionally known technique. Next, the second DNA molecule 23 is added to allow for linking as shown in FIG. 4(b), and thereafter, the unreacted second DNA molecule 23 which was not linked is removed by washing as shown in FIG. 4(c). Furthermore, subsequently, the first DNA molecule 17 is added to allow for linking as shown in FIG. 4(d), and thereafter, the unreacted first DNA molecule 17 which was not linked is removed as shown in FIG. 4(e). The foregoing operations are repeated, and stopped when a desired length was attained. Then, the DNA structure 24 obtained as shown in FIG. 4(f) is detached from the substrate 25.

In the steps of forming the DNA structure 24, any function that is different from each other may be imparted to the first DNA molecule 17 and the second DNA molecule 23 to be the constitutive molecules of the DNA structure 24. For example, a different metal ion may be coordinated to the quadruplex structural part 17a or 23a, or a different functional organic molecule may be bound thereto.

From the foregoing, a DNA structure in which two kinds of the DNA molecules each having an intermolecular antiparallel quadruplex structural part are linked is obtained. Although a DNA structure in which two kinds of the DNA molecules are linked, and the method of the production of the same are described above, a DNA structure in which three or more kinds of the DNA molecules each having an intermolecular antiparallel quadruplex structural part are linked can be also produced by a similar method.

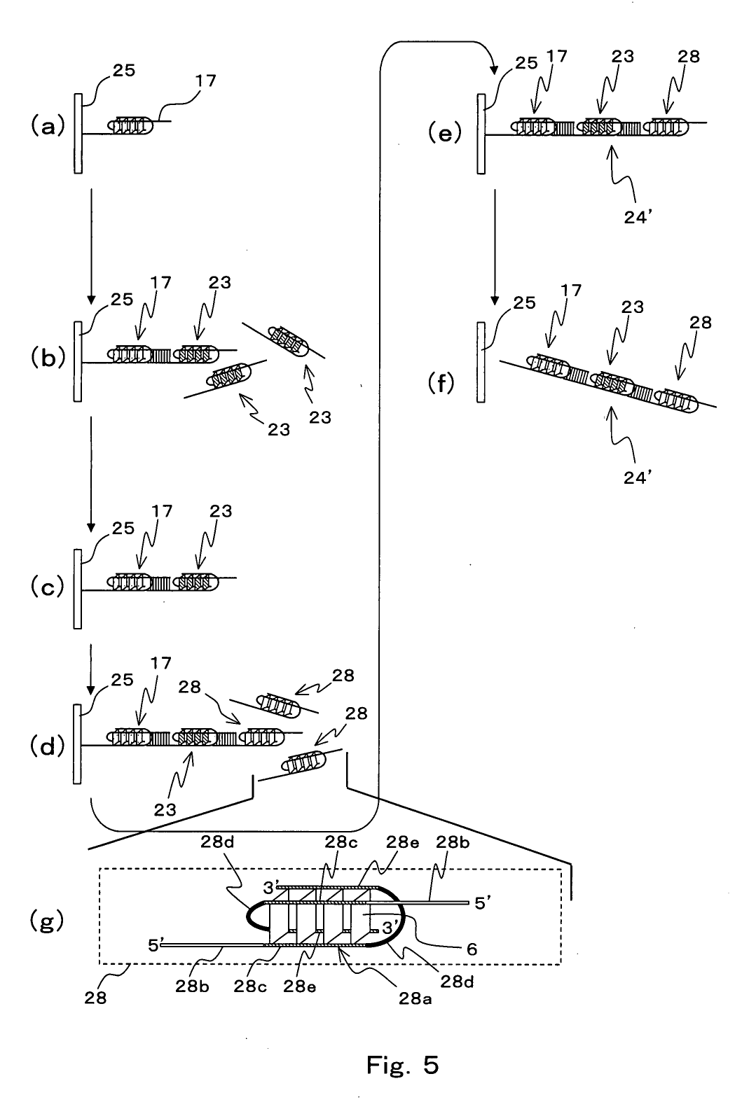

FIG. 5 shows a schematic drawing illustrating a method of obtaining a DNA structure (nanowire) by sequentially linking three kinds DNA molecules each having an intermolecular antiparallel quadruplex structural part. In the method, the first DNA molecule 17 and the second DNA molecule 23 are used as the constitutive molecules in a similar manner to the aforementioned case in which the two kinds of the DNA molecules are linked, and additionally, a third DNA molecule is also used as the constitutive molecule. As shown in FIG. 5(a), the first DNA molecule 17 is previously adsorbed first on the substrate 25 such as gold. Next, the second DNA molecule 23 is added to allow for linking as shown in FIG. 5(b), followed by removing the unreacted second DNA molecule 23 by washing as shown in FIG. 5(c). Additionally, the third DNA molecule 28 of the third kind is then added as shown in FIG. 5(d) in this scheme, although the first DNA molecule 17 is thereafter linked again in FIG. 4.

FIG. 5(g) shows a drawing illustrating an enlarged construction of the third DNA molecule 28. The third DNA molecule 28 includes duplex structure forming sequences 28b at the end, similarly to the first DNA molecule 17 and the second DNA molecule 23, and two molecules of a single stranded DNA including two quadruplex structure forming sequences 28c, 28e and the loop structure forming sequence 28d positioned therebetween are assembled to form G-quartet faces 6 among the four quadruplex structure forming sequences 28c and 28e included in each molecule. Each quadruplex structure forming sequences 28c and 28e may be designed such that the antiparallel quadruplex structural part 28a is formed similarly to the quadruplex structure forming sequences 14, 16, 20 and 22 in the first DNA molecule 17 and the second DNA molecule 23. To this end, it is preferred to design such that at least two or more and five or less G-quartet faces 6 are formed. Furthermore, the loop structure forming sequence 28d may be also designed similarly to the loop structure forming sequences 15 and 21 in the first DNA molecule 17 and the second DNA molecule 23, respectively so as not to inhibit the formation of the antiparallel quadruplex structural part 28a among the quadruplex structure forming sequences. On the other hand, the duplex structure forming sequence 28b in the third DNA molecule 28 is designed so as to interact with the duplex structure forming sequence 19 in the second DNA molecule 23 to form a duplex. Therefore, the third DNA molecule 28 added to the substrate 25 is bound to the second DNA molecule 23 through formation of the duplex by way of the interaction between their duplex structure forming sequences 28b and 19 (FIG. 5(d)).

Following this binding reaction, the unreacted third DNA molecule 28 is removed by washing as shown in FIG. 5(e). Finally, the first DNA molecule 17 is detached from the substrate 25, whereby a DNA structure 24′ in which three kinds of DNA molecules each having an intermolecular antiparallel quadruplex structural part are linked can be obtained as shown in FIG. 5(f).

Furthermore, when the DNA molecule of the fourth kind having an intermolecular antiparallel quadruplex structural part is to be further bound, a fourth DNA molecule is allowed to bind having at its end a duplex structure forming sequence which can form a duplex through interaction with the duplex structure forming sequence of the third DNA molecule in FIG. 5(e), followed by removal of the unreacted fourth DNA molecule. This operation is repeated, and the first DNA molecule 17 is finally detached from the substrate 25 as shown in FIG. 5(f), whereby the DNA structure in which three or more kinds of DNA molecules each having an intermolecular antiparallel quadruplex structural part are sequentially linked can be obtained.

In the foregoing, although the cases in which three or more kinds of DNA molecules each having an intermolecular antiparallel quadruplex structural part are sequentially linked are explained, the order of the linking can be arbitrarily designed.

### Embodiment 2

In this Embodiment 2, a DNA structure in which two kinds of DNA molecules (a first DNA molecule and a second DNA molecule) having an intramolecular antiparallel quadruplex structural part are linked, and a method of the production thereof are explained.

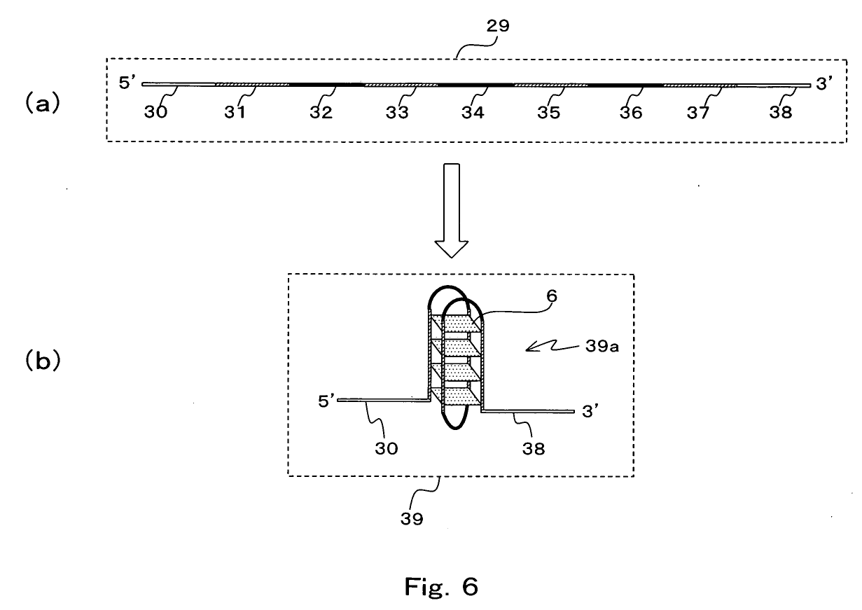

FIG. 6 shows a drawing illustrating steps of forming a first DNA molecule having an intramolecular antiparallel quadruplex structural part. One DNA 29 as shown in FIG. 6(a) associates within the molecule to form a first DNA molecule 39 having an intramolecular antiparallel quadruplex structural part 39a as shown in FIG. 6(b). In the first DNA molecule 39, duplex structure forming sequences 30 and 38 in an unstructured state are extended from two ends of the quadruplex structural part 39a. The first DNA molecule 39 is a monomer DNA molecule. As shown in FIG. 6(a), the DNA 29 is constructed to include, from 5′ end, a duplex structure forming sequence 30, a quadruplex structure forming sequence 31, a loop structure forming sequence 32, a quadruplex structure forming sequence 33, a loop structure forming sequence 34, a quadruplex structure forming sequence 35, a loop structure forming sequence 36, a quadruplex structure forming sequence 37 and a duplex structure forming sequence 38, which are arranged in this order. In the molecule of the DNA 29 having such a construction, interaction among the quadruplex structure forming sequences 31, 33, 35 and 37 occurs, whereby the antiparallel quadruplex structural part 39a is formed to result in formation of the first DNA molecule 39 including the same. In the antiparallel quadruplex structural part 39a, four G bases form a G-quartet face 6 via Hoogsteen hydrogen bondings, and π-π stacking interaction of the G-quartet faces 6 are caused to retain the structure.

In the DNA 29, the base sequence of the quadruplex structure forming sequences 31, 33, 35 and 37 may be designed such that the antiparallel quadruplex structural part 39a is formed. To this end, it is necessary to design so as to form at least two G-quartet faces 6. However, it is more preferred to design so as to form two or more and five or less G-quartet faces 6, because the quadruplex structure forming sequences 31, 33, 35 and 37 are likely to form a parallel quadruplex structure when there are six or more G-quartet faces 6. In the DNA 29, the loop structure forming sequences 32, 34 and 36 are not particularly limited with respect to their base sequence and length, and may be designed so as not to inhibit formation of the quadruplex structural part 39a. Details of the duplex structure forming sequences 30 and 38 will be described herein later. Additionally, the DNA 29 may also include a base sequence other than those described above.

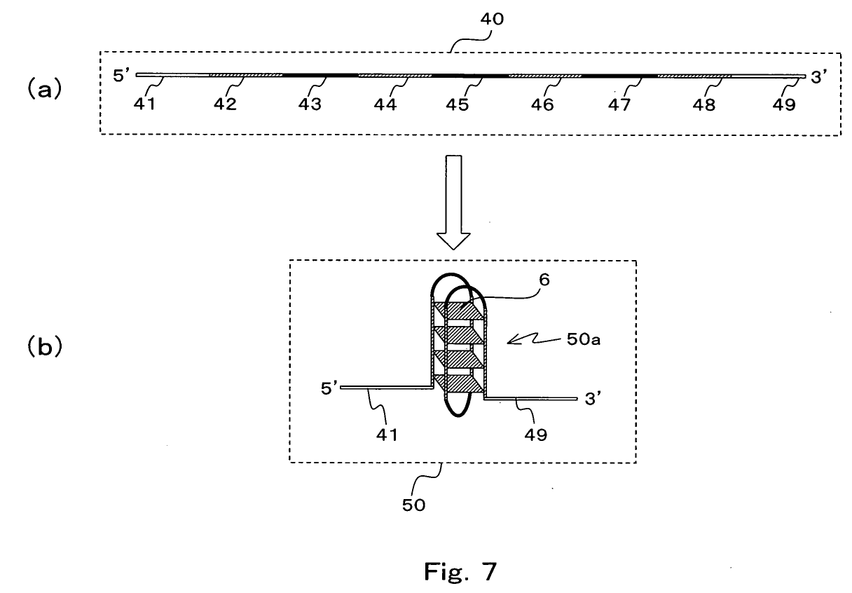

On the other hand, the second DNA molecule having the intramolecular antiparallel quadruplex structural part in this Embodiment 2 also has a construction similar to that of the aforementioned first DNA molecule 29. FIG. 7 shows a drawing illustrating steps of forming a second DNA molecule having an intramolecular antiparallel quadruplex structural part. One DNA 40 as shown in FIG. 7(a) associates within the molecule to form a second DNA molecule 50 having an intramolecular antiparallel quadruplex structural part 50a as shown in FIG. 7(b). In the second DNA molecule 50, duplex structure forming sequences 41 and 49 in an unstructured state are extended from two ends of the quadruplex structural part 50a. The second DNA molecule 50 is a monomer DNA molecule. As shown in FIG. 7(a), the DNA 40 is constructed to include, from 5′ end, a duplex structure forming sequence 41, a quadruplex structure forming sequence 42, a loop structure forming sequence 43, a quadruplex structure forming sequence 44, a loop structure forming sequence 45, a quadruplex structure forming sequence 46, a loop structure forming sequence 47, a quadruplex structure forming sequence 48 and a duplex structure forming sequence 49, which are arranged in this order. In the molecule of the DNA 40 having such a construction, interaction among the quadruplex structure forming sequences 42, 44, 46 and 48 occurs, whereby the antiparallel quadruplex structural part 50a is formed to result in formation of the second DNA molecule 50 including the same. In the antiparallel quadruplex structural part 50a, four G bases form a G-quartet face 6 via Hoogsteen hydrogen bondings, and π-π stacking interaction of the G-quartet faces 6 are caused to retain the structure.

In the DNA 40, the base sequence of the quadruplex structure forming sequences 42, 44, 46 and 48 may be designed similarly to the quadruplex structure forming sequences 31, 33, 35 and 37 of the DNA 29 such that the antiparallel quadruplex structural part 50a is formed. To this end, it is necessary to design so as to form at least two G-quartet faces 6. However, it is more preferred to design so as to form two or more and five or less G-quartet faces 6, because the quadruplex structure forming sequences 42, 44, 46 and 48 are likely to form a parallel quadruplex structure when there are six or more G-quartet faces 6. In the DNA 40, the loop structure forming sequences 43, 45 and 47 are not particularly limited with respect to their base sequence and length, and may be designed so as not to inhibit formation of the quadruplex structural part 50a. Details of the duplex structure forming sequences 41 and 49 will be described herein later. Additionally, the DNA 40 may also include a base sequence other than those described above.

The duplex structure forming sequence 30 of the DNA 29 and the duplex structure forming sequence 41 of the DNA 40 are designed complementary to each other, and the duplex structure forming sequence 38 of the DNA 29 and the duplex structure forming sequence 49 of the DNA 40 are designed complementary to each other. The duplex structure forming sequences 30 and 41, and the duplex structure forming sequences 38 and 49 are acceptable as long as they are designed such that the duplex can be formed by the interaction between each combination, and thus they may not be complementary in the entire sequences. The duplex structure forming sequences 30, 38, 41 and 49 preferably have a base sequence including 10 or more bases in light of structure stability of the DNA structure constructed through linking the first DNA molecule 39 and the second DNA molecule 50.

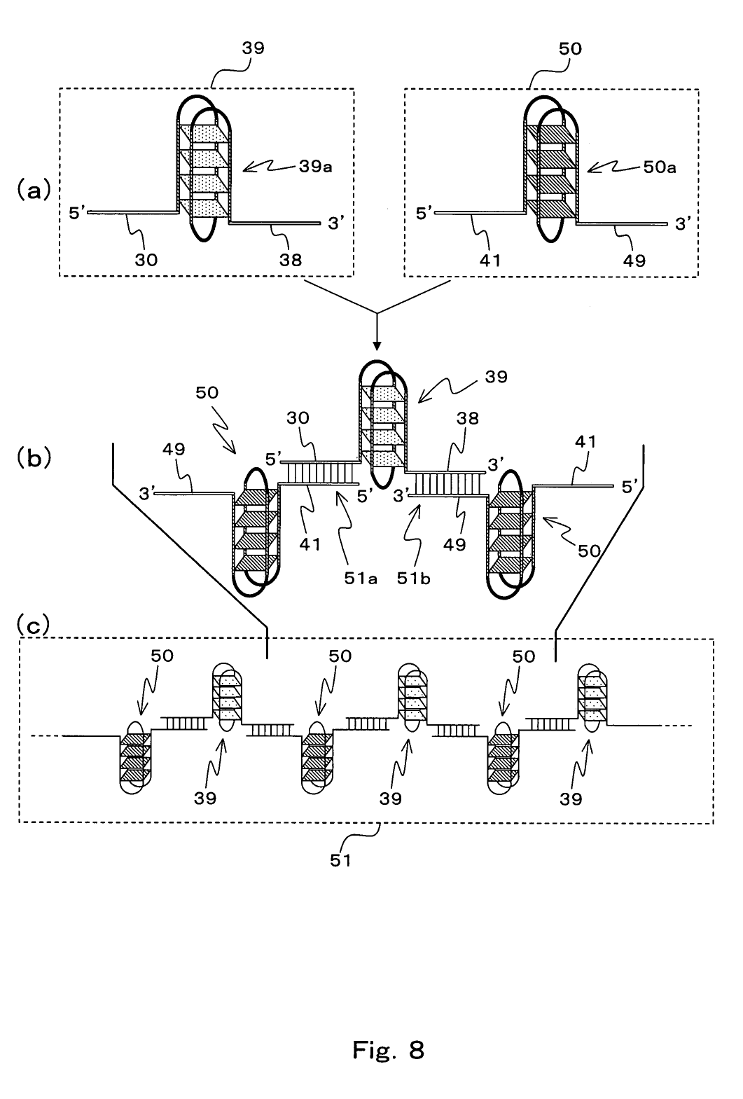

FIG. 8 shows a drawing illustrating steps of forming a DNA structure including a first DNA molecule and a second DNA molecule. When each independently formed first DNA molecule 39 and second DNA molecule 50 each having intramolecular antiparallel quadruplex structural part 39a or 50a as shown in FIG. 8(a) are mixed in an adequate solution, the first DNA molecule 39 and the second DNA molecule 50 are linked in a self-organizing manner via the duplex 51a formed by the interaction of the duplex structure forming sequences 30 and 41, and the duplex 51b formed by the interaction of the duplex structure forming sequences 38 and 49, whereby a DNA structure 51 is formed, as shown in FIG. 8(c). The DNA structure 51 has a construction in which a plurality of the first DNA molecules 39 and the second DNA molecules 50 are linked in one orientation, thereby having a construction as a nanowire. FIG. 8(b) shows a partially enlarged view of the DNA structure 51.

As described above, when the independently formed first DNA molecule 39 and second DNA molecule 50 are merely mixed in the same solution, the DNA structure 51 having a variety of length with both molecules alternately linked would be obtained.

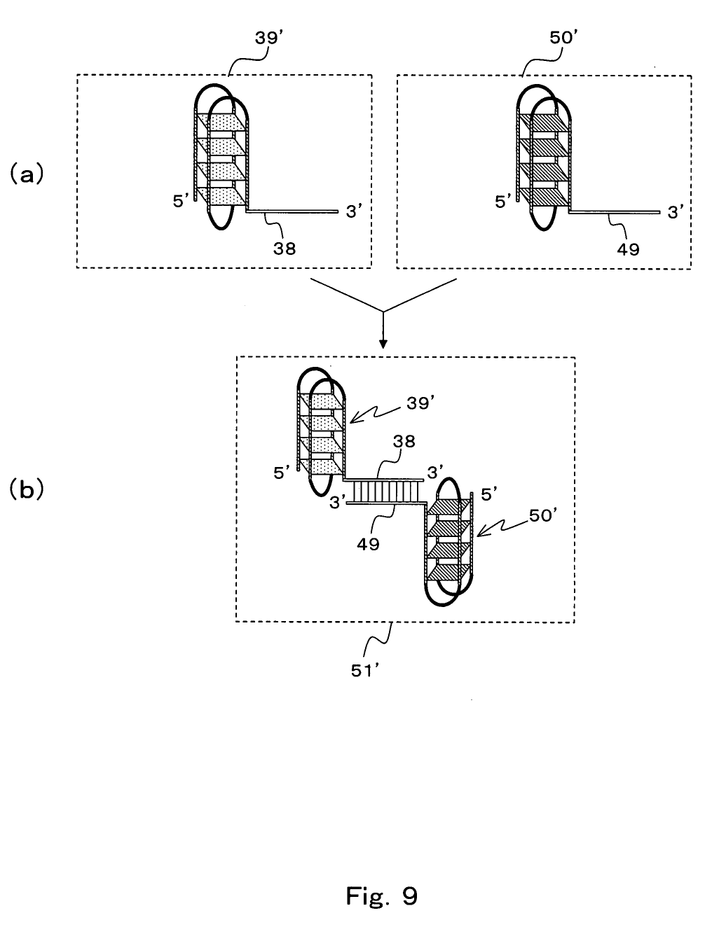

In FIG. 8, both combinations of the duplex structure forming sequences 30 and 41, and the duplex structure forming sequences 38 and 49 are designed to have a complementary relationship, however, they may be also designed such that either one of these combinations has a noncomplementary relationship. Alternatively, the design may be implemented so as not to include at least one of the duplex structure forming sequences 30 and 41, or at least one of the duplex structure forming sequences 38 and 49. Accordingly, one can obtain a DNA structure in which each one molecule of the first DNA molecule 39 and the second DNA molecule 50 are linked. FIG. 9 shows a drawing illustrating steps of forming a DNA structure through linking a first DNA molecule 39′ not having the duplex structure forming sequence 30, and a second DNA molecule 50′ not having the duplex structure forming sequence 41. As shown in FIG. 9(a), by mixing the first DNA molecule 39′ and the second DNA molecule 50′, a DNA structure 51′ in which each one molecule of the first DNA molecule 39′ and the second DNA molecule 50′ are linked as shown in FIG. 9(b) can be obtained.

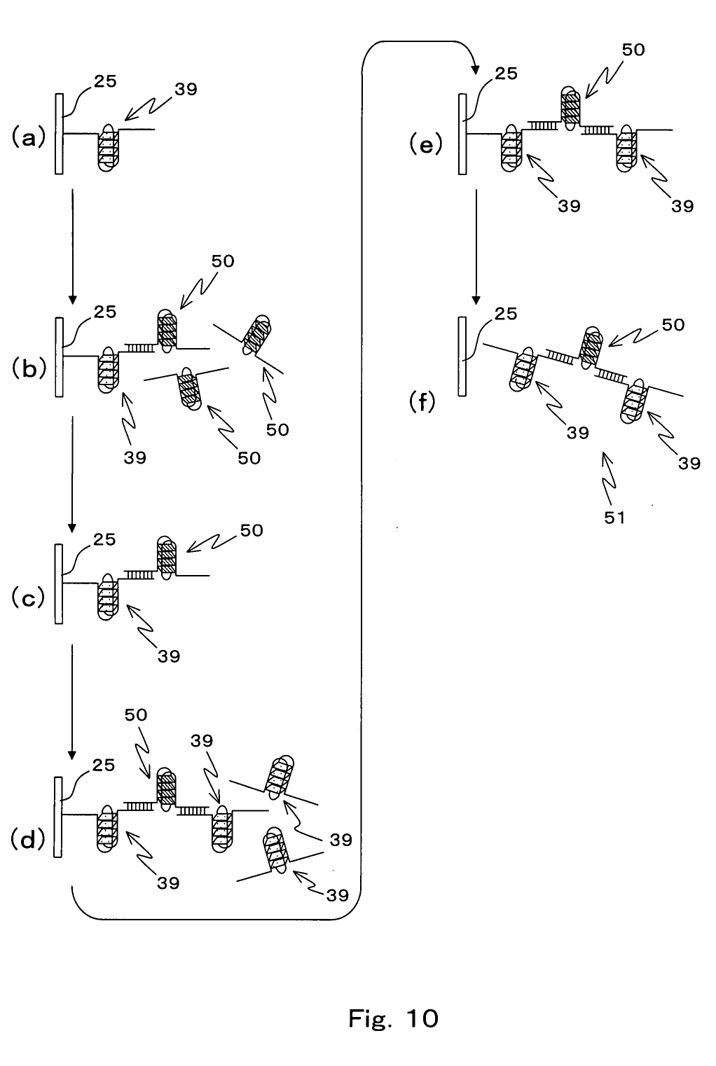

FIG. 10 shows a schematic drawing illustrating a method of obtaining a DNA structure (nanowire) having a desired length by alternately linking a first DNA molecule and a second DNA molecule each having the intramolecular antiparallel quadruplex structural part. As shown in FIG. 10(a), the first DNA molecule 39 is first adsorbed beforehand on a substrate 25 such as gold by a conventionally known technique. Next, the second DNA molecule 50 is added to allow for linking as shown in FIG. 10(b), and thereafter, the unreacted second DNA molecule 50 which was not linked is removed by washing as shown in FIG. 10(c). Furthermore, subsequently, the first DNA molecule 39 is added to allow for linking as shown in FIG. 10(d), and thereafter, the unreacted first DNA molecule 39 which was not linked is removed as shown in FIG. 10(e). The foregoing operations are repeated, and stopped when a desired length was attained. Then, the DNA structure 51 obtained as shown in FIG. 10(f) is detached, from the substrate 25.

In the steps of forming the DNA structure 51, any function that is different from each other may be imparted to the first DNA molecule 39 and the second DNA molecule 50 to be the constitutive molecules of the DNA structure 51. For example, a different metal may be coordinated to the quadruplex structural part 39a or 50a, or a different functional molecule may be bound thereto.

From the foregoing, a DNA structure in which two kinds of the DNA molecules each having an intramolecular antiparallel quadruplex structural part are linked is obtained. A DNA structure in which three or more kinds of the DNA molecules each having an intramolecular antiparallel quadruplex structural part are linked can be obtained by a similar method to that described above. Moreover, a DNA structure in which a plurality of DNA molecules of one kind having an intramolecular antiparallel quadruplex structural part are linked can be also obtained.

In addition, a DNA molecule having an intermolecular antiparallel quadruplex structural part, and a DNA molecule having an intramolecular antiparallel quadruplex structural part, which are independently formed, can be linked in a similar manner to that described above, whereby a DNA structure can be obtained.

### EXAMPLES

Hereinafter, in Examples and Comparative Examples, experiments for producing DNA structures were carried out by allowing two kinds of antiparallel quadruplex DNAs to be linked, which had been each independently formed.

### Comparative Example 1

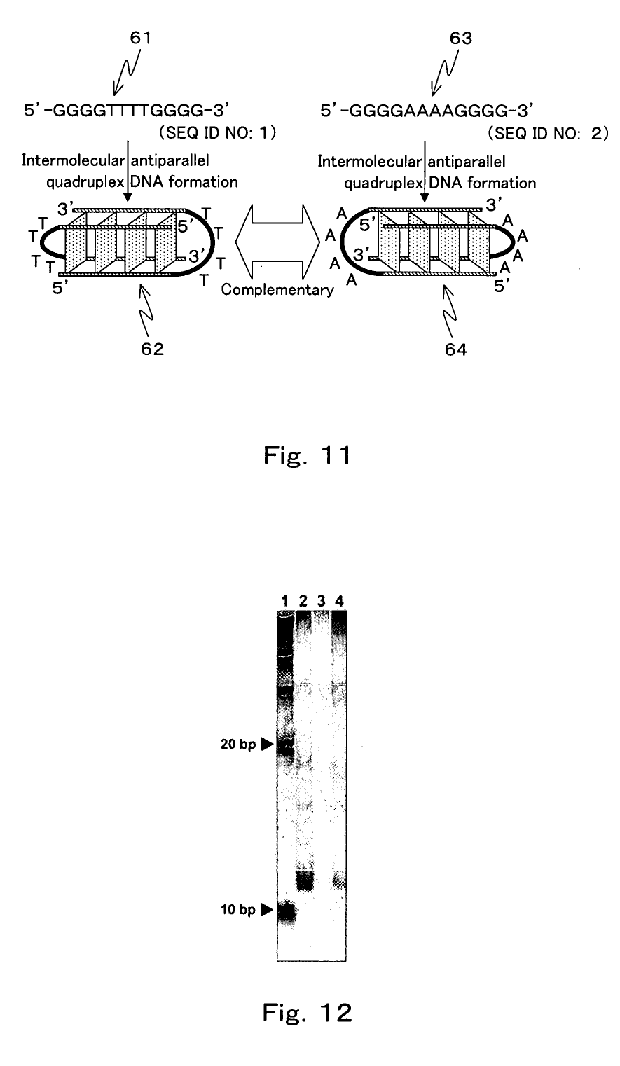

FIG. 11 shows a drawing schematically illustrating an intermolecular antiparallel quadruplex DNA used in Comparative Example 1. In Comparative Example 1, an intermolecular antiparallel quadruplex DNA 62 formed using two strands of DNA 61 (SEQ ID NO: 1) having a loop structure forming sequence consisting of four T bases, and an intermolecular quadruplex DNA 64 formed using two strands of DNA 63 (SEQ ID NO: 2) having a loop structure forming sequence consisting of four A bases were provided as shown in FIG. 11, and both DNAs were mixed in the same solution to study whether they were linked. Since the T base and the A base can form a Watson-Crick base pair, both are expected to form a duplex at the loop site, thereby leading to linking. Specific experimental procedures are as follows.

The used DNA sequences were 5′-GGGGTTTTGGGG-3′ (SEQ ID NO: 1) and 5′-GGGGAAAAGGGG-3′ (SEQ ID NO: 2). The DNA 61 having the former base sequence forms the intermolecular antiparallel quadruplex DNA 62 having a loop consisting of four T bases, and the DNA 63 having the latter base sequence forms the intermolecular quadruplex DNA 64 having four A bases. First, sample solutions obtained by each independently dissolving these two kinds of DNAs 61 and 63 in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹.

Then, these were subjected to circular dichroism (CD) spectrum analysis. As a result, both exhibited positive maximum at a wavelength of 295 nm, and negative maximum at a wavelength of 265 nm. The CD spectra having such features are typically found on antiparallel quadruplex structures. Therefore, it was proven that the aforementioned two kinds of DNAs 61 and 63 formed intermolecular antiparallel quadruplex DNAs 62 and 64, respectively.

Thus, linking of both DNAs that could occur by mixing the solutions containing these two kinds of the intermolecular antiparallel quadruplex DNAs 62 and 64, respectively, was then confirmed. For the confirmation, a native gel electrophoresis was employed. FIG. 12 shows a drawing illustrating results of the electrophoresis. In lane 1 in FIG. 12, a 10-base pairs ladder was electrophoresed which was used as a marker of the electrophoresis. The electrophoresis was carried out with: an annealed sample including the DNA 61 alone in lane 2; an annealed sample including the DNA 63 alone in lane 3; and an annealed sample including the DNA 61 and the DNA 63 mixed at a ratio of 1:1 in lane 4. As a result, the lanes 2 to 4 all exhibited similar electrophoretic migration. Provided two kinds of the intermolecular antiparallel quadruplex DNAs 62 and 64 formed from the DNA 61 and the DNA 63, respectively, were linked, a large structure would be created, whereby the electrophoretic migration would be significantly retarded as compared with unlinked DNAs. However, no difference in the electrophoretic migration was observed between the lanes 2 and 3, and the lane 4. Thus, it was reveled that the linking did not occur through formation of the duplex at the loop site of the antiparallel quadruplex DNAs 62 and 64.

### Comparative Example 2

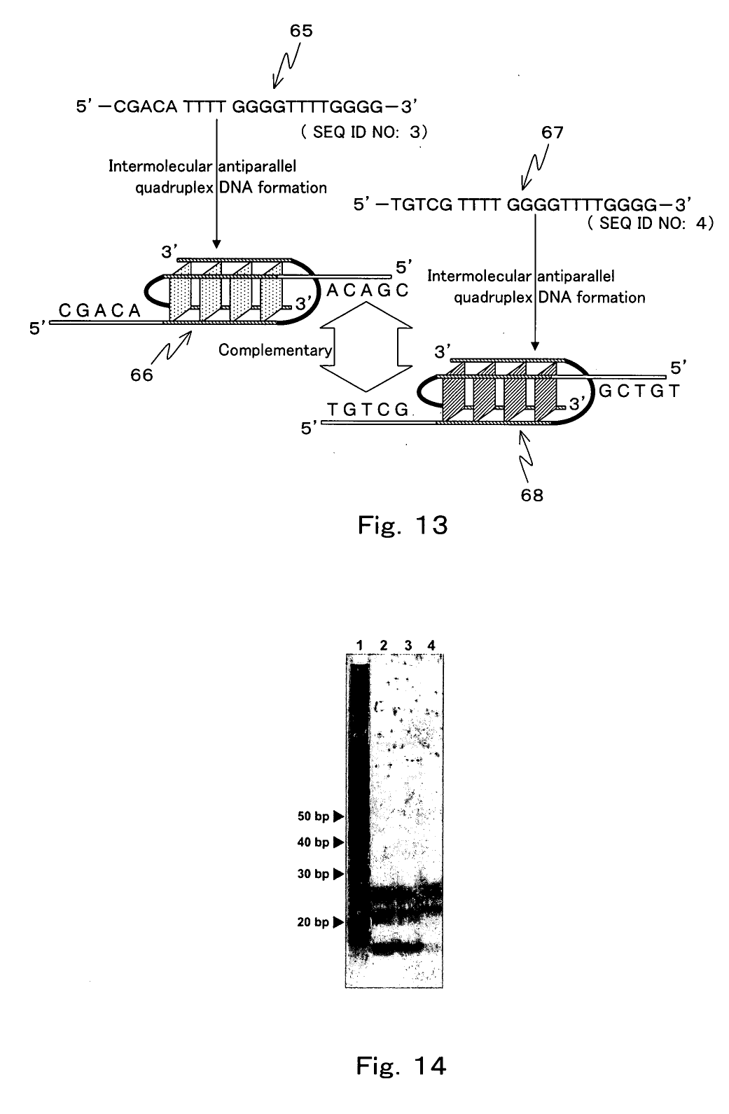

FIG. 13 shows a drawing schematically illustrating an intermolecular antiparallel quadruplex DNA used in Comparative Example 2. In Comparative Example 2, linking of two kinds of intermolecular antiparallel quadruplex DNAs was attempted by providing at the end duplex structure forming sequences that are complementary to one another, as shown in FIG. 13. The used DNAs were DNA 65 (hereinafter, referred to as “N5-1”) having a base sequence of 5′-CGACATTTTGGGGTTTTGGGG-3′ (SEQ ID NO: 3), and DNA 67 (hereinafter, referred to as “N5-2”) having a base sequence of 5′-TGTCGTTTTGGGGTTTTGGGG-3′ (SEQ ID NO: 4), which are believed to be capable of forming intermolecular antiparallel quadruplex DNAs 66 and 68, respectively, as shown in FIG. 13. In addition, since underlined sequence portions are their duplex structure forming sequences, respectively, which are complementary to one another, it is expected that the intermolecular antiparallel quadruplex DNAs are linked which are formed from N5-1 and N5-2 via the duplex formed between these duplex structure forming sequences. Specific experimental procedures are as follows.

First, sample solutions obtained by each independently dissolving single stranded DNAs of the aforementioned N5-1 and N5-2 in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹ to allow for annealing. Then, these were subjected to CD spectrum analysis, and both exhibited positive maximum at a wavelength of 295 nm, and negative maximum at a wavelength of 265 nm. The CD spectra having such features are typically found on antiparallel quadruplex structures. Therefore, it was proven that the aforementioned DNA chains of N5-1 and N5-2 formed intermolecular antiparallel quadruplex DNAs, respectively.

Thus, linking of both DNAs that could occur by mixing the solutions containing these two kinds of the intermolecular antiparallel quadruplex DNAs formed from N5-1 and N5-2 was then confirmed. For the confirmation, a native gel electrophoresis was employed. FIG. 14 shows a drawing illustrating results of the electrophoresis. In lane 1 in the Figure, a 10-base pairs ladder was electrophoresed which was used as a marker of the electrophoresis. The electrophoresis was carried out with: an annealed sample including N5-1 alone in lane 2; an annealed sample including N5-2 alone in lane 3; and an annealed sample including N5-1 and N5-2 mixed at a ratio of 1:1 in lane 4. As a result, the lanes 2 to 4 all exhibited similar electrophoretic migration. Provided two kinds of the intermolecular antiparallel quadruplex DNAs formed from N5-1 and N5-2, respectively, were linked, a large wire structure would be created, whereby the electrophoretic migration would be significantly retarded as compared with unlinked DNAs. Therefore, from the foregoing results, it was unexpectedly proven that linking of the intermolecular antiparallel quadruplex DNAs via each duplex structure forming sequences did not occur.

### Example 1

In Example 1, linking of two kinds of intermolecular antiparallel quadruplex DNAs was attempted by providing at the end duplex structure forming sequences that are complementary to one another, similarly to the aforementioned Comparative Example 2. Although the duplex structure forming sequence included five bases in the Comparative Example 2, ten bases were included in Example 1. The used DNAs were DNA (hereinafter, referred to as “N10-1”) having a base sequence of 5′-CGACATCGCTTTTTGGGGTTTTGGGG-3′ (SEQ ID NO: 5), and DNA (hereinafter, referred to as “N10-2”) having a base sequence of 5′-AGCGATGTCGTTTTGGGGTTTTGGGG-3′ (SEQ ID NO: 6), which are believed to be capable of forming intermolecular antiparallel quadruplex DNAs, respectively. In addition, since underlined sequence portions are their duplex structure forming sequences, respectively, which are complementary to one another, it is expected that the intermolecular antiparallel quadruplex DNAs be linked which are formed from N10-1 and N10-2 via the duplex formed between these duplex structure forming sequences. Specific experimental procedures are as follows.

First, sample solutions obtained by each independently dissolving single stranded DNAs of the aforementioned N10-1 and N10-2 in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹ to allow for annealing. Then, linking of both DNAs that could occur by mixing these two solutions was then confirmed. For the confirmation, CD spectrum analysis and native gel electrophoresis were carried out. In addition, similar experiment was also carried out using 100 mM LiCl in place of 100 mM NaCl. Li ion has been previously known to inhibit formation of the quadruplex DNAs.

Results of the CD spectra are explained first. FIG. 15 shows a drawing illustrating CD spectra obtained in the aforementioned experiment. The CD spectrum was obtained under conditions to give the mixed solution containing 100 mM NaCl or LiCl at 4° C. As also described in Comparative Examples 1 and 2, when the DNA chain forms the antiparallel quadruplex structure, CD spectrum will exhibit a positive maximum at a wavelength of 295 nm, and a negative maximum at a wavelength of 265 nm. Meanwhile, when the DNA chain forms the duplex, CD spectrum will exhibit a positive maximum at a wavelength of 260 nm, and a negative maximum at a wavelength of 240 nm. Thus, when the CD spectrum of the mixed solution of N10-1 and N10-2 in the presence of 100 mM LiCl was analyzed, typical spectrum of the duplex DNA was observed exhibiting a positive maximum at a wavelength of around 260 nm, and a negative maximum at a wavelength of around 240 nm. Since the Li ion has been known to inhibit formation of the quadruplex DNA according to findings hitherto as described above, it would be proper that this CD spectrum is based on the duplex formed with each of the duplex structure forming sequences of N10-1 and N10-2.

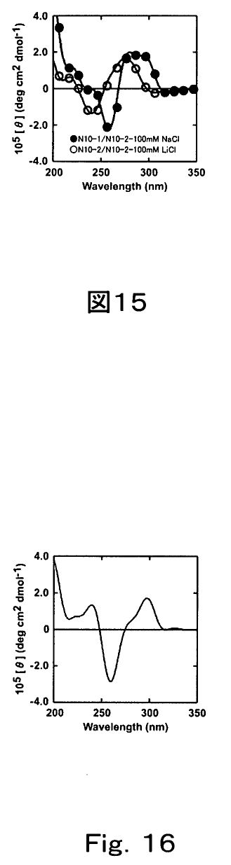

To the contrary, the CD spectrum of the mixed solution of N10-1 and N10-2 in the presence of 100 mM NaCl exhibited a positive peak at a wavelength of around 270 nm, and a negative peak at around 260 nm, and a shoulder at around 290 nm. Provided this spectrum suggests a state in which the intermolecular antiparallel quadruplex DNA consisting of N10-1 and the intermolecular antiparallel quadruplex DNA consisting of N10-2 are linked via each duplex structure forming sequence, the difference spectrum obtained by subtracting the aforementioned CD spectrum in the presence of LiCl from this CD spectrum would derive the CD spectrum of the antiparallel quadruplex DNA. Accordingly, the difference spectrum was obtained. FIG. 16 shows a drawing illustrating the difference spectrum. As shown in FIG. 16, the spectrum derived from the antiparallel quadruplex was obtained exhibiting a positive maximum at a wavelength of around 295 nm, and a negative maximum at a wavelength of around 265 nm. The foregoing results suggest that “duplex+quadruplex structures” were formed in the presence of Na ion, while an “only duplex structure” was formed in the presence of the Li ion.

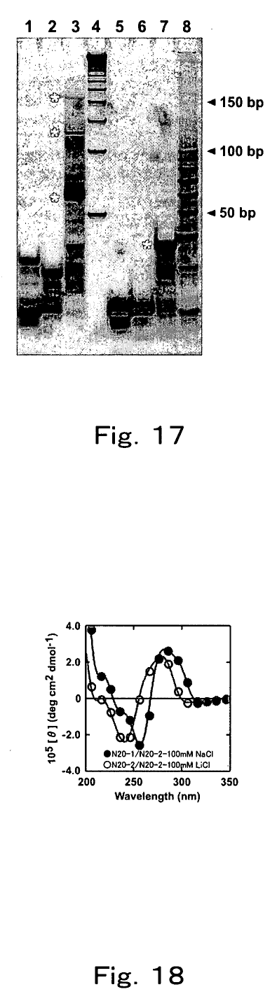

Next, results of the native gel electrophoresis will be explained. FIG. 17 shows a drawing illustrating results of electrophoresis obtained in the aforementioned experiment. The electrophoresis was carried out with: an annealed sample containing N10-1 alone in the presence of 100 mM NaCl in lane 1; an annealed sample containing N10-2 alone in the presence of 100 mM NaCl in lane 2; an annealed mixture sample of N10-1 and N10-2 in the presence of 100 mM NaCl in lane 3; an annealed sample containing N10-1 alone in the presence of 100 mM LiCl in lane 5; an annealed sample containing N10-2 alone in the presence of 100 mM LiCl in lane 6; and an annealed mixture sample of N10-1 and N10-2 in the presence of 100 mM LiCl in lane 7. In lane 4 and lane 8, a 50-base pairs ladder and a 10-base pairs ladder were used as a marker of the electrophoresis. As a result, the annealed mixture sample of N10-1 and N10-2 in the presence of 100 mM NaCl exhibited formation of a polymer structure with retarded electrophoretic migration. In contrast, the annealed mixture sample of N10-1 and N10-2 in the presence of 100 mM LiCl did not form a polymer as found in the presence of the Na ion. Accordingly, it was indicated that a DNA structure in which intermolecular antiparallel quadruplex DNAs independently formed from N10-1 and N10-2, respectively, were linked via the duplex formed between each duplex structure forming sequences was formed only in the presence of the Na ion.

### Example 2

In Example 2, linking of two kinds of intermolecular antiparallel quadruplex DNAs was attempted by providing at the end duplex structure forming sequences that are complementary to one another, similarly to the Comparative Example 2 and Example 1. Although the duplex structure forming sequence included five bases in the Comparative Example 2, and the duplex structure forming sequence included ten bases in Example 1, twenty bases were included in this Example 2. The used DNAs were DNA (hereinafter, referred to as “N20-1”) having a base sequence of 5′-CGACATCGCTCAGCCAGACATTTTGGGGTTTTGGGG-3′ (SEQ ID NO: 7), and DNA (hereinafter, referred to as “N20-2”) having a base sequence of 5′-TGTCTGGCTGAGCGATGTCGTTTTGGGGTTTTGGGG-3′ (SEQ ID NO: 8), which are believed to be capable of forming intermolecular antiparallel quadruplex DNAs, respectively. In addition, since underlined sequence portions are their duplex structure forming sequences, respectively, which are complementary to one another, it is expected that the intermolecular antiparallel quadruplex DNAs be linked which are formed from N20-1 and N20-2 via the formation of these duplexes. Specific experimental procedures are as follows.

First, sample solutions obtained by each independently dissolving the single stranded DNAs of the aforementioned N20-1 and N20-2 in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹ to allow for annealing. Then, linking of both DNAs that could occur by mixing these two solutions was then confirmed. For the confirmation, CD spectrum analysis and native gel electrophoresis were carried out. In addition, similar experiment was also carried out using 100 mM LiCl in place of 100 mM NaCl. The Li ion has been previously known to inhibit formation of the quadruplex DNAs.

Results of the CD spectra are explained first. FIG. 18 shows a drawing illustrating CD spectra obtained in the aforementioned experiment. The CD spectrum was obtained under conditions to give the mixed solution containing 100 mM NaCl or LiCl at 4° C. As also described in Comparative Examples 1 and 2, when the DNA chain forms the antiparallel quadruplex, CD spectrum will exhibit a positive maximum at a wavelength of 295 nm, and a negative maximum at a wavelength of 265 nm. Meanwhile, when the DNA chain forms the duplex, CD spectrum will exhibit a positive maximum at a wavelength of 260 nm, and a negative maximum at a wavelength of 240 nm. Thus, when the CD spectrum of the mixed solution of N20-1 and N20-2 in the presence of 100 mM LiCl was analyzed, typical spectrum of the duplex DNA was observed exhibiting a positive maximum at a wavelength of around 260 nm, and a negative maximum at a wavelength of around 240 nm. Since the Li ion has been known to inhibit formation of the quadruplex DNA according to findings hitherto as described above, it would be proper that this CD spectrum is based on the duplex formed with each of the duplex structure forming sequences of N20-1 and N20-2.

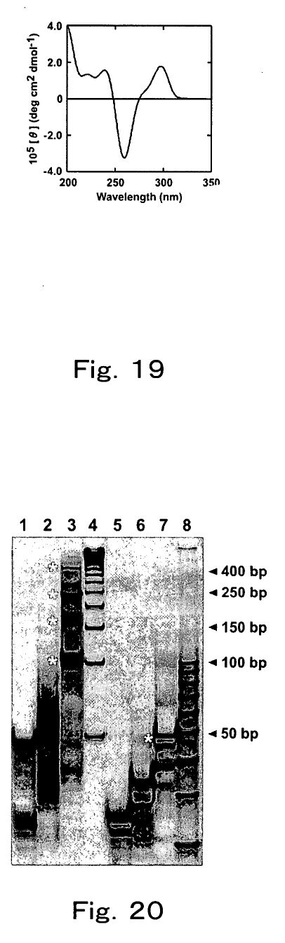

To the contrary, the CD spectrum of the mixed solution of N20-1 and N20-2 in the presence of 100 mM NaCl exhibited a positive peak at a wavelength of around 270 nm, and a negative peak at around 260 nm, and a shoulder at around 290 nm. Provided this spectrum suggests a state in which the intermolecular antiparallel quadruplex DNA consisting of N20-1 and the intermolecular antiparallel quadruplex DNA consisting of N20-2 are linked via each duplex structure forming sequence, the difference spectrum obtained by subtracting the aforementioned CD spectrum in the presence of LiCl from this CD spectrum would derive a typical CD spectrum of the antiparallel quadruplex DNA. Accordingly, the difference spectrum was obtained. FIG. 19 shows a drawing illustrating the difference spectrum. As shown in FIG. 19, the spectrum derived from the antiparallel quadruplex was obtained exhibiting a positive maximum at a wavelength of around 295 nm, and a negative maximum at a wavelength of around 265 nm. The foregoing results suggest that “duplex+quadruplex structures” were formed in the presence of Na ion, while an “only duplex structure” was formed in the presence of the Li ion.

Next, results of the native gel electrophoresis will be explained. FIG. 20 shows a drawing illustrating results of electrophoresis obtained in the aforementioned experiment. The electrophoresis was carried out with: an annealed sample containing N20-1 alone in the presence of 100 mM NaCl in lane 1; an annealed sample containing N20-2 alone in the presence of 100 mM NaCl in lane 2; an annealed mixture sample of N20-1 and N20-2 in the presence of 100 mM NaCl in lane 3; an annealed sample containing N20-1 alone in the presence of 100 mM LiCl in lane 5; an annealed sample containing N20-2 alone in the presence of 100 mM LiCl in lane 6; and an annealed mixture sample of N20-1 and N20-2 in the presence of 100 mM LiCl in lane 7. In lane 4 and lane 8, a 50-base pairs ladder and a 10-base pairs ladder were used as a marker of the electrophoresis. As a result, the annealed mixture sample of N20-1 and N20-2 in the presence of 100 mM NaCl exhibited formation of a polymer structure with retarded electrophoretic migration. In contrast, the annealed mixture sample of N20-1 and N20-2 in the presence of 100 mM LiCl did not form a polymer as found in the presence of the Na ion. Accordingly, it was indicated that a DNA structure in which intermolecular antiparallel quadruplex DNAs independently formed from N20-1 and N20-2, respectively, were linked through the formation of the duplex between each duplex structure forming sequences was formed only in the presence of the Na ion.

As in the foregoing, it was revealed from the results of Comparative Examples 1 and 2, and Examples 1 and 2 that intermolecular antiparallel quadruplex DNAs are linked when duplex structure forming sequences to be complementary to one another are provided at each ends of independently formed two kinds of intermolecular antiparallel quadruplex DNAs, and when the duplex structure forming sequence includes a length of 10 or more bases.

### Example 3

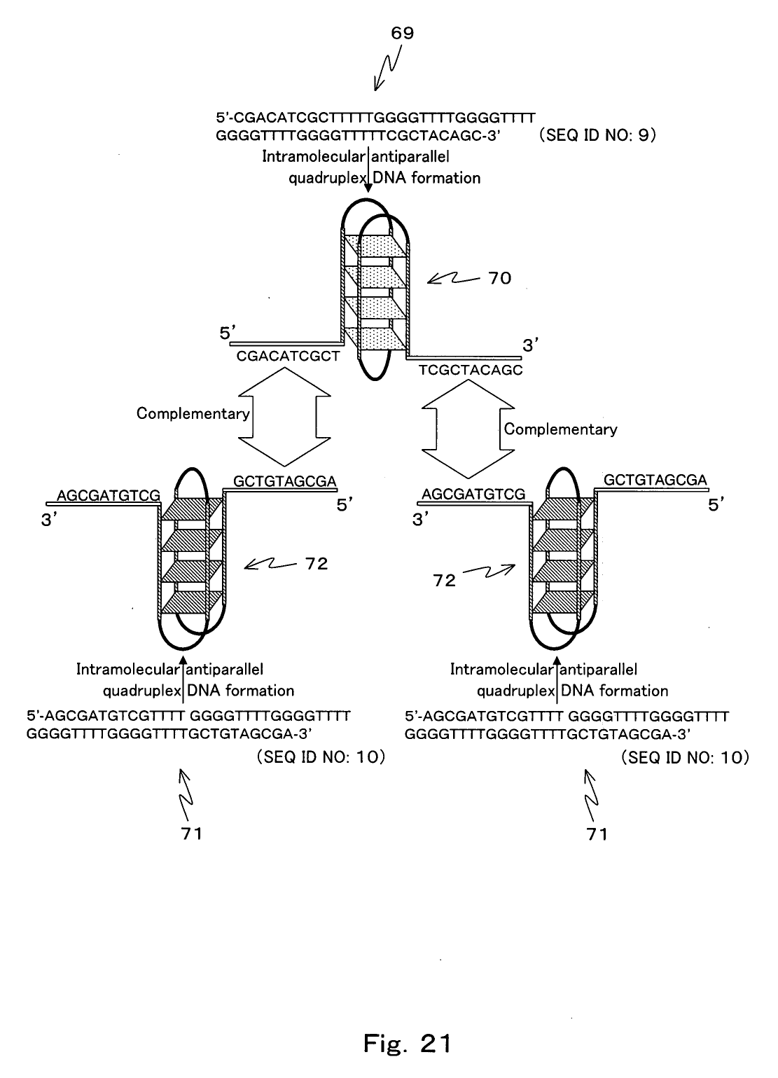

FIG. 21 shows a drawing schematically illustrating an intramolecular antiparallel quadruplex DNA used in Example 3. In Example 3, linking of independently formed two kinds of intramolecular antiparallel quadruplex DNAs was attempted via the duplex structure forming sequences provided at the ends, as shown in FIG. 21. The used DNAs were DNA 69 (hereinafter, referred to as “M10-1”) having a base sequence of 5′-CGACATCGCTTTTTGGGGTTTTGGGGTTTTGGGGTTTTGGGGTTTTTCGCTACAGC-3′ (SEQ ID NO: 9), and DNA 71 (hereinafter, referred to as “M10-2”) having a base sequence of 5′-AGCGATGTCGTTTTGGGGTTTTGGGGTTTTGGGGTTTTGGGGTTTTGCTGTAGCGA-3′ (SEQ ID NO: 10), which are complementary to one another at underlined sequences, and at double-underlined sequences.

Sample solutions obtained by dissolving each of these in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹ to allow for annealing. These annealed samples were each subjected to CD analysis, and both exhibited positive maximum at a wavelength of 295 nm, and negative maximum at a wavelength of 265 nm. Accordingly, these were proven to form intramolecular antiparallel quadruplex DNAs 70 and 72.

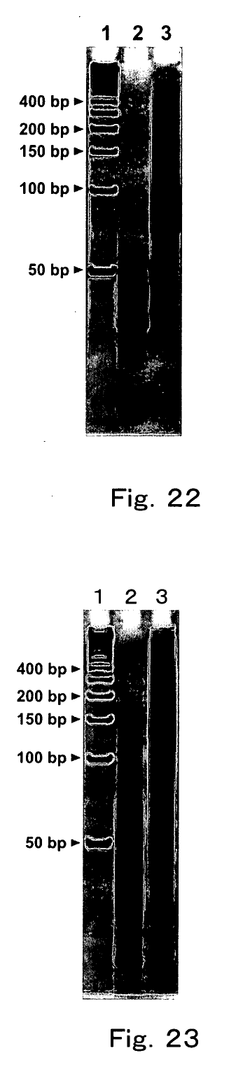

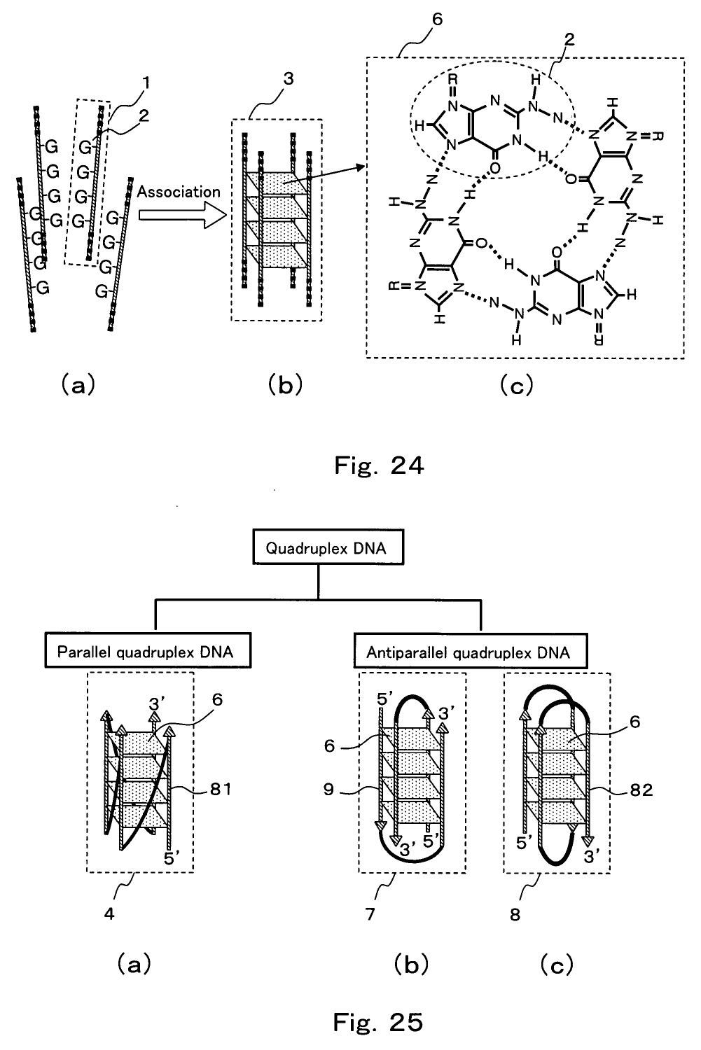

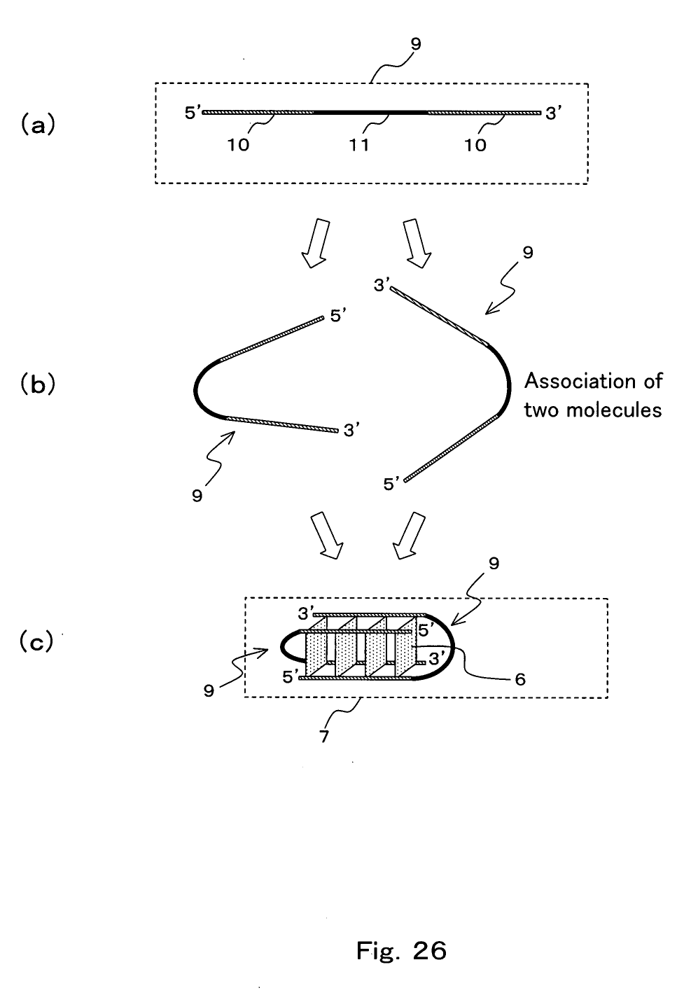

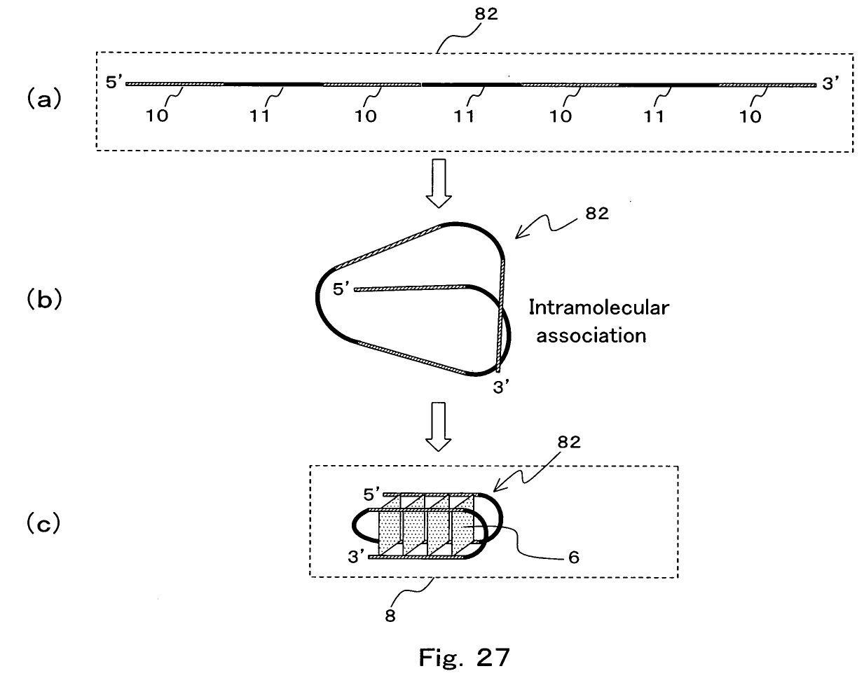

Thus, a sample obtained by mixing these two sample solutions at a ratio of 1:1 was analyzed by native gel electrophoresis. FIG. 22 shows a drawing illustrating results of the electrophoresis obtained by this experiment. In lane 1, a 50-base pairs ladder was electrophoresed which was used as a marker of the electrophoresis. The electrophoresis was carried out with: an annealed sample including M10-1 alone in lane 2; and a mixed solution including M10-1 and M10-2 mixed at a ratio of 1:1 in lane 3. As a result, widespread distribution of the electrophoretic migration of the mixed sample was ascertained. Such widespread distribution of the electrophoretic migration is believed to result from formation of the polymer, thereby suggesting that each intramolecular antiparallel quadruplex DNAs formed from M10-1 and M10-2 are linked via the duplex between the underlined and double-underlined sequences.

### Example 4

In Example 4, linking of independently formed two kinds of intramolecular antiparallel quadruplex DNAs was attempted via the duplex structure forming sequences provided at the ends, similarly to the above Example 3. Although the duplex structure forming sequence in Example 3 included 10 bases, 20 bases were included in this Example. The used DNAs were a DNA (hereinafter, referred to as “M20-1”) having a base sequence of 5′-CGACATCGCTCAGCCAGACATTTTGGGGTTTTGGGGTTTTGGGGTTTTGGGGTTTTACAGACCGACTCGCTACAGC-3′ (SEQ ID NO: 11), and a DNA (hereinafter, referred to as “M20-2”) having a base sequence of 5′-TGTCTGGCTGAGCGATGTCGTTTTGGGGTTTTGGGGTTTTGGGGTTTTGGGGTTTTGCTGTAGCGAGTCGGTCTGT-3′ (SEQ ID NO: 12), which are complementary to one another at underlined sequences, and at double-underlined sequences. Sample solutions obtained by dissolving each of these in a buffer solution, pH 7.0 (50 mM Tris-HCl) containing 100 mM NaCl were incubated at 95° C. for 5 min, and the temperature was lowered from 95° C. to 4° C. at a cooling speed of 0.2° C. min⁻¹ to allow for annealing. These annealed samples were each subjected to CD analysis, and both exhibited positive maximum at a wavelength of 295 nm, and negative maximum at a wavelength of 265 nm. Accordingly, these were proven to form intramolecular antiparallel quadruplex DNAs.

Thus, a sample obtained by mixing these two sample solutions at a ratio of 1:1 was analyzed by native gel electrophoresis. FIG. 23 shows a drawing illustrating results of the electrophoresis obtained by this experiment. In lane 1, a 50-base pairs ladder was electrophoresed which was used as a marker of the electrophoresis. The electrophoresis was carried out with: an annealed sample including M20-1 alone in lane 2; and a mixed solution including M20-1 and M20-2 mixed at a ratio of 1:1 in lane 3. As a result, widespread distribution of the electrophoretic migration of the mixed sample was ascertained. Such widespread distribution of the electrophoretic migration is believed to result from formation of the polymer, thereby suggesting that each intramolecular antiparallel quadruplex DNAs formed from M20-1 and M20-2 are linked via the duplex between the underlined and double-underlined sequences.

According to the present invention, a DNA structure in which multiple quadruplex structural parts are linked, and a method of the production of the same are provided.

The DNA structure produced by the present invention can include metal ions such as K ion, Na ion or the like arranged in its structure, and thus it is useful in novel functional nanowires such as molecular wires, molecular magnets, nanoalloys, nanocatalyst wires and the like.

From the foregoing description, many modifications and other embodiments of the present invention will be apparent to persons skilled in the art. Therefore, the foregoing description should be construed as merely illustrative examples, which were provided for the purpose of teaching persons skilled in the art best modes for carrying out the present invention. Details of the structure and/or function of the present invention can be substantially altered without departing from the spirit of the invention.

What is claimed is:

1. A method of producing a DNA structure in which multiple quadruplex DNAs are linked,
the method comprising (a) a step of mixing multiple DNA molecules having an antiparallel quadruplex structural part, and at least two single stranded sticky ends extended from the end of the quadruplex structural part,
wherein the single stranded sticky end of the each DNA molecule has a base sequence that can form a duplex through interaction with the single stranded sticky end of other DNA molecule.

2. The method of producing a DNA structure according to claim 1 wherein the DNA molecules to be mixed in the step (a) comprise at least two kinds of molecules, i.e., a first DNA molecule and a second DNA molecule,
wherein one single stranded sticky end of the first DNA molecule, and one single stranded sticky end of the second DNA molecule have a base sequence that can form a duplex through interaction, and wherein other single stranded sticky end of the first DNA molecule, and other single stranded sticky end of the second DNA molecule have a base sequence that can form a duplex through interaction.

3. The method of producing a DNA structure according to claim 2 wherein the first DNA molecule and the second DNA molecule are a monomer DNA molecule having the quadruplex structural part each formed intramolecularly.

4. The method of producing a DNA structure according to claim 2 wherein the first DNA molecule and the second DNA molecule are a dimer DNA molecule having the quadruplex structural part each formed intermolecularly.

5. The method of producing a DNA structure according to claim 1 wherein the single stranded sticky end has a base sequence comprising 10 or more bases.

6. The method of producing a DNA structure according to claim 1 wherein the quadruplex structural part comprises two or more and five or less G-quartet faces formed by a hydrogen bonding among four guanine bases.

7. A DNA structure in which multiple DNA molecules having an antiparallel quadruplex structural part are linked via a junction part, wherein:
the DNA molecule has at least two single stranded sticky ends extended from the end of the quadruplex structural part;
the single stranded sticky end of the each DNA molecule has a base sequence that can form a duplex through interaction with the single stranded sticky end of other DNA molecule; and
the junction part is a duplex formed through the interaction of the single stranded sticky ends of adjacent two DNA molecules.


SEQUENCE LISTING (22 sequences)

SEQ ID NO: 1 - DNA, 12 bp, Artificial Sequence
  Oligonucleotide for Quadruplex DNA structure
ggggttttgggg

SEQ ID NO: 2 - DNA, 12 bp, Artificial Sequence
  Oligonucleotide for Quadruplex DNA structure
ggggaaaagggg

SEQ ID NO: 3 - DNA, 21 bp, Artificial Sequence
  Oligonucleotide for quadruplex DNA structure
cgacattttggggttttgggg

SEQ ID NO: 4 - DNA, 21 bp, Artificial Sequence
  Oligonucleotide for quadruplex DNA structure
tgtcgttttggggttttgggg

SEQ ID NO: 5 - DNA, 26 bp, Artificial Sequence
  Oligonucleotide for quadruplex DNA structure
cgacatcgctttttggggttttgggg

SEQ ID NO: 6 - DNA, 26 bp, Artificial Sequence
  Oligonucleotide for Quadruplex DNA structure
agcgatgtcgttttggggttttgggg

SEQ ID NO: 7 - DNA, 36 bp, Artificial Sequence
  Oligonucleotide for quadruplex DNA structure
cgacatcgctcagccagacattttggggttttgggg

SEQ ID NO: 8 - DNA, 36 bp, Artificial Sequence
  Oligonucleotide for quadruplex DNA structure
tgtctggctgagcgatgtcgttttggggttttgggg

SEQ ID NO: 9 - DNA, 56 bp, Artificial Sequence
  Oligonucleotide for quadruplex DNA structure
cgacatcgctttttggggttttggggttttggggttttggggtttttcgctacagc

SEQ ID NO: 10 - DNA, 56 bp, Artificial Sequence
  Oligonucleotide for Quadruplex DNA structure
agcgatgtcgttttggggttttggggttttggggttttggggttttgctgtagcga

SEQ ID NO: 11 - DNA, 76 bp, Artificial Sequence
  Oligonucleotide for Quadruplex DNA structure
cgacatcgctcagccagacattttggggttttggggttttggggttttggggttttacagaccgactcgctacagc

SEQ ID NO: 12 - DNA, 76 bp, Artificial Sequence
  Oligonucleotide for Quadruplex DNA structure
tgtctggctgagcgatgtcgttttggggttttggggttttggggttttggggttttgctgtagcgagtcggtctgt

SEQ ID NO: 13 - DNA, 10 bp, Artificial Sequence
  Known oligonucleotide for Quadruplex DNA structure
gggttttggg

SEQ ID NO: 14 - DNA, 12 bp, Artificial Sequence
  Known oligonucleotide for Quadruplex DNA structure
ggggttttgggg

SEQ ID NO: 15 - DNA, 12 bp, Artificial Sequence
  Known oligonucleotide for Quadruplex DNA structure
gggcttttgggc

SEQ ID NO: 16 - DNA, 11 bp, Artificial Sequence
  Known oligonucleotide for Quadruplex DNA
gcggtttgcgg

SEQ ID NO: 17 - DNA, 12 bp, Artificial Sequence
  Known oligonucleotide for Quadruplex DNA structure
aaggttttaagg

SEQ ID NO: 18 - DNA, 9 bp, Artificial Sequence
  Known oligonucleotide for Quadruplex DNA structure
gggttcagg

SEQ ID NO: 19 - DNA, 28 bp, Artificial Sequence
  Known oligonucleotide for Quadruplex DNA structure
ggggttttggggttttggggttttgggg

SEQ ID NO: 20 - DNA, 24 bp, Artificial Sequence
  Known oligonucleotide for Quadruplex DNA structure
ttggggttggggttggggttgggg

SEQ ID NO: 21 - DNA, 15 bp, Artificial Sequence
  Known oligonucleotide for Quadruplex DNA structure
ggttggtgtggttgg

SEQ ID NO: 22 - DNA, 22 bp, Artificial Sequence
  Known Oligonucleotide for Quadruplex DNA structure
agggttagggttagggttaggg